SEQUENCE LISTING (13 sequences)

SEQ ID NO: 1 - DNA, 33 bp, Lactococcus lactis
  source 1..33: mol_type = other DNA
ggaattcgatttagatgccataggaggattttc

SEQ ID NO: 2 - DNA, 33 bp, Lactococcus lactis
  source 1..33: mol_type = other DNA
gatgaatatcgtacatcctccaattttttaatg

SEQ ID NO: 3 - DNA, 37 bp, Lactococcus lactis
  source 1..37: mol_type = other DNA
aagcttgataaaacaagaaaatattcatgaaattcag

SEQ ID NO: 4 - DNA, 39 bp, Lactococcus lactis
  source 1..39: mol_type = other DNA
ggaggatgtacgatattcatcttaagaaaaatcaaaagc

SEQ ID NO: 5 - DNA, 26 bp, Lactococcus lactis
  source 1..26: mol_type = other DNA
catctaaatcgaattcctgcagcccg

SEQ ID NO: 6 - DNA, 30 bp, Lactococcus lactis
  source 1..30: mol_type = other DNA
tcttgttttatcaagcttatcgataccgtc

SEQ ID NO: 7 - DNA, 27 bp, Lactococcus lactis
  source 1..27: mol_type = other DNA
ttggattagctgcggcatattttatcg

SEQ ID NO: 8 - DNA, 27 bp, Lactococcus lactis
  source 1..27: mol_type = other DNA
ccttgttgaccataatgcaaagcaggt

SEQ ID NO: 9 - DNA, 30 bp, Lactococcus lactis
  source 1..30: mol_type = other DNA
aaaatatagaaggagactattgcaaatagc

SEQ ID NO: 10 - DNA, 30 bp, Lactococcus lactis
  source 1..30: mol_type = other DNA
aaaaattaatggccatcgttggtagttctc

SEQ ID NO: 11 - DNA, 29 bp, Lactococcus lactis
  source 1..29: mol_type = other DNA
tctgtgcagcagaaatggcgacggttgaa

SEQ ID NO: 12 - DNA, 30 bp, Lactococcus lactis
  source 1..30: mol_type = other DNA
tccccataaatttttctttttcactcgcat

SEQ ID NO: 13 - DNA, 30 bp, Lactococcus lactis
  source 1..30: mol_type = other DNA
cggttattcctcaaaaagacttatcattaa

(19) United States
(12) Patent Application Publication
(10) Pub. No.: US 2026/0137734 A1
(43) Pub. Date: May 21, 2026

(54) LACTOCOCCUS LACTIS STRAINS FOR THE PREVENTION AND/OR THE TREATMENT OF VISCERAL PAIN

(71) Applicants: Institut National De Recherche Pour L'Agriculture, L'Alimentation Et L'Environnement, Paris (FR); Institut National Des Sciences Appliquees De Toulouse, Toulouse (FR); Centre National De La Recherche Scientifique, Paris (FR); Ecole D'Ingenieurs De Purpan, Toulouse (FR); Ecole Nationale Veterinaire De Toulouse, Toulouse (FR); Universite Toulouse III - Paul Sabatier, Toulouse (FR)

(72) Inventors: Muriel COCAIGN-BOUSQUET, Mons (FR); Hélène EUTAMENE, Balma (FR); Muriel MERCIER BONIN, Toulouse (FR); Vassilia THEODOROU, Portet-Sur-Garonne (FR); Valérie LAROUTE, Toulouse (FR); Marie-Line DAVERAN-MINGOT, Castanet (FR)

(21) Appl. No.: 19/457,518

(22) Filed: Jan. 23, 2026

Related U.S. Application Data
(63) Continuation of application No. 17/310,295.

(30) Foreign Application Priority Data
Feb. 1, 2019 (EP) 19305125.7

(51) Int. Cl. A61K 35/744; A23L 29/00; A61P 29/00

(52) U.S. Cl. CPC A61K 35/744; A23L 29/065; A61P 29/00; A23V 2400/231

(57) ABSTRACT

The present invention relates to a Lactococcus lactis strain or a cell fraction thereof for use in the prevention and/or treatment of visceral pain.

### CROSS-REFERENCE TO RELATED APPLICATIONS

This application is a continuation of U.S. application Ser. No. 17/310,295, filed Jul. 27, 2021, which is a national phase entry of PCT/EP2020/052466, filed on Jan. 31, 2020, which claims the benefit of priority of EP patent application Ser. No. 19/305,125.7, filed on Feb. 1, 2019, the contents of which being hereby incorporated by reference in its entirety for all purposes.

### REFERENCE TO A SEQUENCE LISTING

The Sequence Listing written in file 644944_SeqListing. XML is 12,759 bytes in size, was created on Jan. 21, 2026, and is hereby incorporated by reference.

### FIELD

The present invention relates to Lactococcus lactis strains which have the ability to reduce pain, in particular visceral pain; these strains are useful for the prevention and/or the treatment of visceral pain in an individual. The present invention also relates to new strains among these Lactococcus lactis strains and compositions containing them.

### BACKGROUND

Visceral pain is pain associated with the viscera, which encompass the internal organs of the body. Here the term of viscera includes all the gastro-intestinal tract from the oral cavity to the anus. These organs also include, for example, the heart, lungs, reproductive organs, bladder, ureters, the organs of the digestive tract, preferably tongue, abdominal gut, liver, pancreas, spleen, and kidneys. Visceral pain is characterized by a vague, diffuse, dull, aching pain, which is localized but can have a tendency to radiate abdomen and/or back. It can be accompanied by a feeling of malaise, and, when severe, it induces strong autonomic phenomena such as sweating, vasomotor responses, bradycardia, nausea and vomiting. It is usually felt in the midline and deep in the body.

There are a variety of conditions in which abdominal or visceral pain may exist. For example, pancreatitis pain, labor pain, pain from abdominal surgery associated with ileus, pain in irritable bowel syndrome, abdominal pain in non-ulcer dyspepsia, or in dysmenorrhea. Likewise, liver pain, kidney pain, epigastric pain, pleural pain, and painful biliary colic, appendicitis pain may all be considered to be visceral pain. Substernal pain or pressure from early myocardial infarction is also visceral. Further, burning mouth syndrome (BMS) is a chronic idiopathic disorder characterized by burning sensation or dysesthesia in the tongue and other oral sites without clear etiology even if gastric origins are suspected. This BMS is accompanied by dry mouth, tingling, or dysgeusia. Considering the peripheral nervous alterations identified in BMS patients, some clinical trials have demonstrated the efficacy of topical GABA-A binding receptor ligands on BMS symptoms (de Castro et al., 2014). Diseases of the stomach, duodenum or colon can cause visceral pain. Commonly encountered gastrointestinal (GI) disorders that cause visceral pain include functional bowel disorder (FBD) and inflammatory bowel disease (IBD). These GI disorders include a wide range of disease states that are currently only moderately controlled, including, with respect to FBD, gastro-esophageal reflux, dyspepsia, irritable bowel syndrome (IBS) and functional abdominal pain syndrome (FAPS), and, with respect to IBD, Crohn's disease, ileitis and ulcerative colitis, all of which regularly produce visceral pain.

According to Rome IV criteria, irritable bowel syndrome (IBS) is a functional gastrointestinal disorder with unknown etiology, affecting 10-20% of adults in most countries. IBS results in various symptoms that strongly affect the patient's quality of life (Dean et al., 2005). It has been shown that stress is an important factor in the onset, maintenance and deterioration of IBS (Chang et al., 2011), which results in significant morbidity and health care costs. IBS is a disorder of the brain-gut axis and clinical symptoms include abdominal pain and changes in bowel habits (i.e., constipation, diarrhea) without structural abnormalities (Camilleri et al., 2012). A subset of IBS patients, varying from 30% to 40%, were reported to exhibit enhanced sensitivity to colonic distension, which is noticeable through their reduced threshold for pain, increased intensity of sensations and/or exaggerated viscerosomatic referral in response to colonic distension (Mayer et al., 1994, Mayer et al., 2008, Bouin et al., 2001, Zhou et al., 2010). Thus, visceral hypersensitivity has been proposed as a key clinical marker accounting for the severity of bowel movements, bloating and abdominal pain symptoms experienced by IBS patients (Kuiken et al., 2005, Posserud et al., 2007, Camilleri et al., 2008). For a better understanding of the IBS pathophysiology, acute and chronic stress animal models were developed mimicking IBS features, such as changes in visceral sensitivity and gut transit time (Gué et al., 1997). In rat, acute restraint stress associated with visceral hypersensitivity to rectal distension is linked to central release of corticotrophin releasing factor (CRF) (Gué et al., 1997). Current pharmacological treatments for these disorders (IBS and BMS) are to date mainly symptom-driven and their efficacy is generally low (Craig et al., 2018).

It appears from the foregoing that there is a need for alternatives to pharmacological agents for the prevention and/or the treatment of visceral pain.

Some experimental evidence suggests that probiotics would be an effective treatment option to modulate abdominal pain in particular in IBS patients (Moayyedi et al., 2010, Hungin et al., 2013, Ford et al., 2014, Didari et al. 2015); however, clinical results differ considerably among studies due to the strains of probiotics used, mixtures and dosages of these strains, as well as treatment duration and subtypes of IBS patients (Mazurak et al., 2015).

Recently, the human gut commensal Bifidobacterium dentium, producing γ-aminobutyric acid (GABA), was shown to modulate sensory neuron activity in a rat fecal retention model of visceral hypersensitivity (Pokusaeva et al., 2017). Moreover, the Escherichia coli strain Nissle 1917 was demonstrated to produce the C12AsnGABAOH lipopeptide, able to inhibit visceral sensitivity in mice (Pérez-Berezo et al., 2017). However, no proof of the efficacy of the probiotic itself was given, only the analgesic properties of the C12AsnGABAOH lipopeptide was shown.

Thus, it remains useful to identify new therapeutic approaches to reduce visceral pain and that are easy to produce with a low production cost.

### BRIEF SUMMARY

In this context, the Inventors focused on the study of the lactic acid bacteria, Lactococcus lactis. While the Lactococcus lactis subsp. lactis strains are known to produce GABA in vitro (Nomura et al., 1999) and are theoretically, genetically able to express the pyridocal-5′-phosphate (PLP)-dependent enzyme glutamate decarboxylase (GAD), the enzyme which converts the glutamate in GABA, their effective capacity to express GAD and produce GABA is very aleatory for reasons that are not known. Indeed, the Inventors showed that despite the strong similarity of the gad operon involved in GABA biosynthesis between L. lactis NCDO2727 and NCDO2118 strains, GABA production under similar culture conditions was very different with a very weak amount of GABA produced by the NCDO2727 strain (GABA concentration always below 0.2 mM in FIG. 2A). In a same way, a high GAD activity was obtained in the NCDO2118 strain, while in the NCDO2727 strain, the amount was extremely low (Table 3).

Frequently encountered in dairy products, L. lactis is one of the most ingested bacteria (Mills et al., 2010, Laroute et al., 2017). This bacterium is commonly found in dairy products such as cheese or dairy fermented products, including fermented milk.

Although not considered as a commensal bacterium, L. lactis was found to persist transiently in the gut, depending on the strain under study (Wang et al., 2011, Radziwill-Bienkowska et al., 2016, Zhang et al., 2016).

The Inventors identified new L. lactis strains able to produce high amounts of GABA, i.e., at least 10 mM of γ-aminobutyric acid (GABA) in a time of culture lower or equal of 24 hours, and they have shown that these strains possess the surprising and unexpected ability to reduce visceral pain. Thus, these L. lactis strains represent new agents for preventing and/or treating visceral pain.

Moreover, the Inventors have demonstrated that a L. lactis stain, NCDO2727, which produces low amount of GABA, do not possess such advantageous properties.

Accordingly, an object of the present invention is a Lactococcus lactis strain able to produce at least 10 mM of γ-aminobutyric acid (GABA) in a time of culture lower or equal to 24 hours, for use in the prevention and/or the treatment of visceral pain, in an individual.

Preferably, the Lactococcus lactis strain of the present invention is able to produce at least 10 mM of GABA, more preferably 20 mM of GABA, still more preferably 40 mM of GABA and most preferably at least 60 mM of GABA in a time of culture lower or equal to 24 hours (FIG. 2A).

The ability to produce GABA may be assessed by culturing a Lactococcus lactis strain in a medium M17 supplemented with glutamate, arginine, glucose and NaCl in a fermenter. In the first hours of culture, the pH is maintained at 6.6, GAD is induced and accumulated. At 7-8 hours of culture, the strains are collected for individual treatment then pH is decreased at 4.6 to quantify GABA production rate. The concentration of GABA is measured over time until 24 hours.

The preparation of culture media and the definition of the culture conditions allowing the growth and the induction of GAD expression in the strains of Lactococcus lactis of the present invention are well known by the person skilled in the art; such culture medium may be adapted to each specific L. lactis strain; for example, for the strain CNCM I-5388, the addition of arginine and NaCl to a basic M17 culture medium has been proven to induce the expression of GAD; other strains such as CNCM I-5386 are able to enhance the GAD expression at a level similar to NCDO2118 when cultured on this M17 culture medium without addition of any specific compound.

An individual according to the invention is a mammal or a bird; for example, the individual may be an animal from a livestock breeding, such as poultry, or a pet; according to a specific embodiment, the individual is a human.

Preferably, the strain L. lactis of the present invention is characterized in that the activity of the pyridocal-5′-phosphate (PLP)-dependent enzyme glutamate decarboxylase (GAD) produced by said strain is at least 20 μmole/min·mg of protein in a time of culture comprised between 7 hours and 8 hours.

Preferably, the activity of the GAD produced by Lactococcus lactis strain in the present invention is at least 20 μmole/min·mg of protein, more preferably 40 μmole/min·mg, still more preferably 60 μmole/min·mg and in an increasing order at least 100, at least 200, at least 300, at least 400, at least 500, at least 600, at least 700 μmole/min·mg and most preferably at least 820 μmole/min·mg in a time of culture comprised between 7 hours and 8 hours.

Preferably, the strain L. lactis of the present invention is characterized in that the GABA production rate at pH 4.6 by said strain is at least of 1 mmole/h/g of cell dry weight in a time of culture comprised between 8.5 hours and 11.5 hours.

Preferably, the specific production rate of GABA in the present invention is at least 1 mmole/h/g of cell dry weight, more preferably 3 mmole/h/g of cell dry weight and most preferably at least 7 mmole/h/g of cell dry weight in a time of culture comprised between 8.5 hours and 11.5 hours.

Methods for assessing the GAD activity are known in the art as shown in Example 1. The GABA production rate is a specific rate calculated between 8.5 hours and 11.5 hours (mean value).

The strain L. lactis in the present invention can be selected in the group consisting of: CNCM I-5388, CNCM I-5386 and NCDO2118, preferably between CNCM I-5388 and CNCM I-5386, and more preferably said L. lactis strain is the strain CNCM I-5388.

The strains CNCM I-5388 and CNCM I-5386 were deposited according to the Budapest Treaty, at CNCM (Collection Nationale de Cultures de Microorganismes, 25 rue du Docteur Roux, Paris) on Nov. 29, 2018 for CNCM I-5386 and on Dec. 13, 2018 for CNCM I-5388.

The L. lactis strains of the present invention are of a particular interest in preventing or treating visceral pain.

The prophylactic and therapeutic effect of the L. lactis strains of the invention is obtained in presence of free glutamate; for example, the amount of free glutamate is about 4 mg per 10⁹ cfu of L. lactis and per day or is about 17 mg/kg bw/day.

As demonstrated in Example 2, free glutamate per se does not produce any beneficial or unwanted effect on basal and stress-induced colorectal activity.

Depending of the continent and diet culture, classic diet provides different levels of free glutamate (average population intakes of free glutamate range from 5.5 mg/kg bw/day (elderly Austrians) to 37 mg/kg bw/day (toddlers in Belgium); high-level intakes range up to 82 mg/kg bw/day for other children in Belgium, 77 mg/kg bw/day for Danish infants, and 56 mg/kg bw/day for other children in Bulgaria; moreover, from natural free glutamate and dietary protein, a range of 81 to 155 mg/kg bw/day can be ingested in adults. David R. Tennant, Ann. Nutr. Metab. 2018; 73:21-28). Therefore, according to these data, the Inventors considered that L. lactis strains of the invention can be administered alone, i.e. without the addition of free glutamate to exert an efficient prevention or a reduction of abdominal or visceral pain.

Alternatively, in case of free glutamate-free diet or diet especially poor in free glutamate, the L. lactis strains of the invention may be administered together with free glutamate. Accordingly, in a specific embodiment, another object of the present invention is the combination of strain L. lactis of the present invention and of between 10 and 20 mg/kg bw/day, in particular 17 mg/kg bw/day of free glutamate for use in the prevention and/or the treatment of visceral pain, in an individual.

For the different aspects of present invention, visceral pain may be caused by an injury, an infection, an inflammation, a disease or by a thermal stimulus, a mechanical stimulus, an electric stimulus a chemical stimulus or a radioactive stimulus.

Visceral pain in the invention is in particular those observed in chronic abdominal and pelvic pain disorders, including irritable bowel syndrome (IBS) but also burning mouth syndrome (BMS). IBS patients exhibit significantly lower response thresholds to provocative stimuli (i.e., rectal distension), complain of increased sensitivity during normal organ function, and present increased tenderness in expanded areas of somatic (abdominal) referral (i.e., both visceral and somatic hypersensitivity). Functional visceral (gastro-intestinal) disorders resulted from altered central nervous system processing and/or dysregulated central modulation. In this cascade of events, visceral inputs of primary afferent nociceptors located in the Dorsal Root Ganglia (DRG) are responsible for changes in the perception of pain. However, all chronic diseases such as hepatitis, peptic ulcer. IBD (Crohn's disease and ulcerative colitis). IBS in which abdominal and/or back pain is a principal complaint and all the perceived sensations are initiated by activity in peripheral sensory (afferent) neurons (Keszthelyi D. et al., 2012). Regarding Burning mouth syndrome (BMS) that is an important chronic pain disorder that affects more than 1 million people in the United States (Miziara et al., 2009). BMS is characterized by both positive (burning pain, dysgeusia and dysesthesia) and negative (loss of taste and paraesthesia) sensory symptoms involving the lips and tongue, mainly the tip and anterior two-thirds. BMS may be classified into three types (Sun et al., 2013). In Type 1, patients are free of pain on waking but experience increasing symptoms as the day goes on. In Type 2, patients have continuous pain throughout the day: this type accounts for 55% of patients and has the strongest association with psychological disorders (Coculescu et al., 2014). In Type 3, patients have intermittent symptoms with pain-free periods during the day. Moreover, these BMS patients frequently have depression, anxiety, sometimes diabetes, and even nutritional/mineral deficiencies.

According to an embodiment of the invention, visceral pain is caused by a gastrointestinal disorder, such as an Irritable Bowel Syndrome or Burning Mouth Syndrome characterized by burning of the oral mucosa in the absence of underlying dental or medical causes. This is also the case with a functional and organic dyspepsia, narcotic bowel syndrome, IBD, colonic cancer, chronic pancreatitis, diverticulitis, appendicitis, ovarian cyst.

It is known that visceral pain is increased in various psychological stress conditions (acute and chronic).

An acute partial restraint stress increases visceral sensitivity (hypersensitivity) and also reduces the threshold of the sensitivity (allodynia).

According to an embodiment of the invention, the L. lactis strains in the present invention are useful when visceral pain is linked to psychological stress events and/or anxiety. The model of the psychological stress events and/or anxiety is an acute partial restraint stress (PRS).

PRS is considered as a mild non-ulcerogenic model (Williams et al., 1987) and consists of under light anesthesia with ethyl ether, constraining animal body movements. For this, the upper forelimbs of the animal are taped up to the thoracic trunk in order to constrain animal body movements during 2 hours. Rats are then replaced in their home cages. In rats PRS is reflected by both an increase of adrenocorticotropic hormone (ACTH) and corticosterone plasmatic concentrations (Sun et al., 2006), associated with visceral hypersensitivity to rectal distension linked to central release of corticotrophin releasing factor (CRF), (Gue et al., 1997), as well as increase in gut permeability (Ait-Belgnaoui et al., 2005).

Indeed, the Inventors showed that L. lactis NCDO2118, producing high level of GABA, prevents visceral hypersensitivity in an acute-stress rat model, whereas L. lactis NCDO2727, producing low level of GABA, is not able to induce such an effect.

Further, the NCDO2118 ΔgadB mutant strain, unable to produce GABA, failed to prevent visceral hypersensitivity in the same model.

In one aspect of the invention, the L. lactis strains of the present invention are characterized in that the effect produced by these strains is obtained from 10 days, more preferably from 7 days, still more preferably from 5 days and most preferably from 4 days of the treatment of visceral pain linked to psychological stress events and/or anxiety.

Indeed, the Inventors showed that the beneficial effect produced by CNCM I-5388 is obtained from 5 days of the treatment of visceral pain linked to an acute stress, and the effect produced by NCDO2118 is obtained from 10 days of such treatment.

Another aspect of the invention is the Lactococcus lactis strain as defined above, for use in the prevention and/or the treatment of visceral pain due to burning mouth syndrome (BMS).

An object of the present invention is also a cell fraction obtained from the Lactococcus lactis strain as defined above, for use in the prevention and/or the treatment of visceral pain and in particular for use in the prevention and/or the treatment of visceral pain linked to psychological stress events and/or anxiety.

Said cell fraction is in particular preparations containing bacterial enzymes, especially GAD, obtained from cultures of L. lactis of the invention. It may also be culture supernatants or fractions of these strains. The cell fractions can be prepared according to the methods known for the person skilled in the art. In a non-limited way, these methods generally include a step of lysis of the bacteria obtained after culture and a step of separation of the fractions containing the membranes of the said bacteria from the total lysate obtained after the lysis step, in particular by centrifugation or filtration.

Another aspect of the invention is the cell fraction obtained from Lactococcus lactis strain as defined above, for use in the prevention and/or the treatment of visceral pain due to burning mouth syndrome (BMS).

An object of the present invention is also a composition comprising the Lactococcus lactis strain according to the present invention or the cell fraction according to the present invention and optionally free glutamate preferably in an amount of between 10 and 20 mg/kg bw/day, in particular of 17 mg/kg bw/day, for use in the prevention and/or the treatment of visceral pain.

In the composition of the invention, said strain can be used in the form of whole bacteria which may be living or dead. Alternatively, said strain can be used in the form of a bacterial lysate. Preferably the bacterial cells are present as living, viable cells.

The composition of the invention can be in any form suitable for administration, in particular oral administration. This includes for instance solids, semi-solids, liquids, and powders.

When the bacteria are in the form of living bacteria, the composition given per day may comprise a content of the L. lactis strain according to the present invention allowing the administration of at least 10⁶ colony forming units (cfu) of said L. lactis strain, preferably at least 10⁷ cfu, still more preferably at least 10⁸ cfu, and most preferably at least 10⁹ cfu.

Another aspect of the invention is the composition comprising the Lactococcus lactis strain according to the present invention or the cell fraction according to the present invention, for use in the prevention and/or the treatment of visceral pain due to burning mouth syndrome (BMS).

An object of the present invention is also a method for preventing and/or treating visceral pain, comprising administering to a mammal in need thereof, a therapeutic amount of the Lactococcus lactis strain of the invention or the cell fraction of the invention, as defined above. Determination of a therapeutic amount is well known from the person skilled in the art, especially in view of the detailed disclosure provided herein.

An object of the present invention is also the use of the Lactococcus lactis strain of the invention or the cell fraction of the invention, as defined above for the manufacture of a medicament for preventing and/or treating visceral pain.

As used herein, the treatment or prevention encompasses inter alia: preventing and/or decreasing visceral pain.

The composition of the present invention can be a pharmaceutical composition or a nutritional composition, including food products such as food supplements and functional food. The food products comprise dairy products and dairy fermented products.

A pharmaceutical composition can be in an oral form such as tablets, capsules, powders, syrups effervescent compositions and sprays. Said pharmaceutical composition can also be in sublingual and buccal forms such as sublingual tablets and solution compositions that are administered under the tongue and buccal tablets that are placed between the cheek and gum.

A food supplement designates a product made from compounds usually used in foodstuffs, but which is in the form of tablets, powder, capsules, potion or any other form usually not associated with aliments, and which has beneficial effects for one's health.

A functional food is an aliment which also has beneficial effects for one's health. In particular, food supplements and functional food can have a physiological effect, protective or curative, against a disease or a pain.

The composition can be in the form of a liquid or present in the form of a dry powder obtained by drying the liquid.

In a preferred embodiment, the product is a fresh product. A fresh product, which has not undergone severe heat treatment steps, has the advantage that the bacterial strains present are in the living form.

The Inventors have also identified two new Lactococcus lactis strains having the specific abilities discussed previously.

Thus, the present invention also provides a Lactococcus lactis strain, characterized in that said strain is selected in the group consisting of: CNCM I-5388, CNCM I-5386.

The present invention also encompasses a cell fraction obtained from the Lactococcus lactis strain selected in the group consisting of: CNCM I-5388, CNCM I-5386.

The present invention also provides a composition comprising said Lactococcus lactis strain as defined above or said cell fraction as defined above.

Said composition can comprise at least 10⁶ colony forming units (cfu), preferably at least 10⁷ cfu, more preferably at least 10⁸ cfu, and most preferably at least 10⁹ cfu per gram dry weight of the composition.

The composition as defined above can be a pharmaceutical composition or a nutritional composition, including food products as defined above.

Said composition can be in the form of a liquid or present in the form of a dry powder obtained by drying the liquid.

In a preferred embodiment, the product is a fresh product.

An object of this invention is related to a pharmaceutical composition comprising the Lactococcus lactis strain or the cell fraction as defined above and a pharmaceutically acceptable vehicle; optionally said composition also comprises free glutamate.

In an embodiment of the invention, the composition as defined above is for use as a medicament. The formulation of said medicament is adapted allowing local treatment in the form of a spray or tablets to melt under the tongue.

Said composition is particularly useful for preventing and/or treating visceral pain.

The present invention further relates to a method of treatment for preventing and/or treating visceral pain as defined above, comprising the administration of the Lactococcus lactis strain or the cell fraction or the composition as defined above to an individual.

The present invention also relates to the Lactococcus lactis strain or the cell fraction of the invention, optionally in association with free glutamate, for the preparation of a medicine for the prevention and/or the treatment of visceral pain as defined above.

### BRIEF DESCRIPTION OF THE SEVERAL VIEWS OF THE DRAWINGS

The present invention will be understood more clearly from the further description which follows, which refers to examples illustrating the capacity of the L. lactis strains in the present invention for reducing visceral pain, as well as to the appended figures.

FIG. 1 shows the organization of the gad cluster in the different strains.

FIG. 2 shows the GABA production (mM) (A) and evolution of biomass (g/L) (B) during growth of L. lactis NCDO2118 (square), CNCM I-5388 (diamond), CNCM I-5386 (triangle) or NCDO2727 (circle) in M17 supplemented with free glutamate (8 g/L), arginine (5 g/L), glucose (45 g/L) and NaCl (300 mM). Independent duplicates (filled symbols: first experiment and open symbols: second experiment).

FIG. 3 shows the effect of 10-day oral administration of free glutamate (2 mg in the diet and 2 mg co-administrated with L. lactis) on and PRS-induced colorectal sensitivity of vehicle-treated rats at all the distension pressures of CRD (from 15 to 60 mmHg). Data are expressed as means±SEM (n=10 for the “vehicle” group (open square, dashed line); n=10 for the “PRS+vehicle” group (black square, solid line); n=11 for the “vehicle+free glutamate” group (black circle, dashed line); n=11 for the “PRS+vehicle+free glutamate” group (black circle, solid line)). *P<0.05; **P<0.01; ***P<0.001 vs. basal values for animals treated with vehicle.

FIG. 4 shows the effect of 10-day oral administration of L. lactis NCDO2118 and L. lactis NCDO2727 on PRS-induced visceral hypersensitivity at all the distension pressures of CRD (from 15 to 60 mmHg). Data are expressed as means±SEM (n=9 for the “vehicle” group (white square, dashed line) and “PRS+vehicle” group (black square, solid line); n=12 for the “PRS+NCDO2118+free glutamate” group (black triangle, solid line); n=8 for the “PRS+NCDO2727+free glutamate” group (black star, solid line)). ***P<0.001 vs. basal values for animals treated with vehicle. ⁺P<0.05; ⁺⁺P<0.01 vs. values for stressed animals treated with vehicle.

FIG. 5 shows the effect of 5-day or 10-day-oral administration of L. lactis NCDO2118 and L. lactis CNCM I-5388 on PRS-induced visceral hypersensitivity at all the distension pressures of CRD (from 15 to 60 mmHg). A. Results obtained with L. lactis NCDO2118 at 5 days. Data are expressed as means±SEM (n=11 for the “vehicle” group (white square, dashed line), n=11 for the “PRS+vehicle” group (black square, solid line) and n=11 for the “PRS+NCDO2118+free glutamate” group (black triangle, solid line)). **P<0.01 significantly different vs. basal values for animals treated with vehicle. B. Results obtained with L. lactis CNCM I-5388 at 5 days. Data are expressed as means±SEM (n=7 for the “vehicle” group (white square, dashed line); n=7 for the “PRS+vehicle” group (black square, solid line); n=7 for the “PRS+CNCM I-5388” group (black/white diamond, dash-dotted line) and “PRS+CNCM I-5388+free glutamate” (black diamond, solid line)). *P<0.05; **P<0.01 vs. basal values for animals treated with vehicle, C. Results obtained with L. lactis NCDO2118 at 10 days. Data are expressed as means±SEM (n=9 for the “vehicle” group (white square, solid line); n=9 for the “PRS+vehicle” group (black square, solid line); n=12 for the “PRS+NCDO2118+free glutamate” group (black triangle, solid line); n=8 for the “PRS+NCDO2118” group (black/white triangle, dash-dotted line)) ***P<0.001 vs. basal values for animals treated with vehicle. ⁺P<0.05; ⁺⁺P<0.01 vs. values for stressed animals treated with vehicle. D. Results obtained with L. lactis CNCM I-5388 at 10 days. Data are expressed as means±SEM (n=7 for the “vehicle” group (white square, dashed line); n=7 for the “PRS+vehicle” group (black square, solid line); n=8 for the “PRS+CNCM I-5388” group (black/white diamond, dash-dotted line) and n=7 for the “PRS+CNCM I-5388+free glutamate” (black diamond, solid line).**P<0.01 vs. basal values for animals treated with vehicle. ⁺P<0.05 vs. values for stressed animals treated with vehicle.

FIG. 6 shows the effect of GABA-producing L. lactis NCDO2118 and the non-GABA producing NCDO2118 ΔgadB mutant (10⁹ cfu per day) on PRS-induced visceral hypersensitivity at all the distension pressures of CRD (from 15 to 60 mmHg). Data are expressed as means±SEM (n=13 for the “vehicle” group (white square, dashed line) and “PRS+vehicle” group (black square, solid line); n=14 for the “PRS+NCDO2118+free glutamate” group (black triangle, solid line); n=13 for the “PRS+NCDO2118 ΔgadB+free glutamate” group (inverted triangle, dash-dotted line)). **P<0.01; ****P<0.0001 vs. basal values for animals treated with vehicle. ⁺⁺P<0.01; ⁺⁺⁺P<0.001 vs. values for stressed animals treated with vehicle ^{S}P<0.05 vs. values for stressed animals treated with NCDO2118.

FIG. 7 shows the effect of 5-day or 10-day-oral administration of L. lactis CNCM I-5388 on PRS-induced visceral hypersensitivity at all the distension pressures of CRD (from 15 to 60 mmHg). A. Results obtained with L. lactis CNCM I-5388 at 5 days. Data are expressed as means±SEM (n=10 for the “vehicle” group (white square, dashed line), n=10 for the “PRS+vehicle” group (black square, solid line), n=10 for the “PRS+CNCM I-5388” group (white ring, solid line), n=9 for the “PRS+CNCM I-5388+free glutamate” group (black ring, solid line), n=9 for the “PRS+CNCM I-5388+free glutamate+inhibitor” group (white rhombus, solid line), n=9 for the “PRS+CNCM I-5388 Δgad mutant+free glutamate” group (black rhombus, solid line). *P<0.05; **P<0.01; ****P<0.0001 vs. basal values for animals treated with vehicle. ⁺⁺⁺P<0.001 vs. values for stressed animals treated with vehicle. B. Results obtained with L. lactis CNCM I-5388 at 10 days. Data are expressed as means±SEM (n=10 for the “vehicle” group ((white square, dashed line), n=10 for the “PRS+vehicle” group (black square, solid line), n=9 for the “PRS+CNCM I-5388” group (white ring, solid line), n=9 for the “PRS+CNCM I-5388+free glutamate” group (black ring, solid line), n=9 for the “PRS+CNCM I-5388+free glutamate+inhibitor” group (white rhombus, solid line), n=9 for the “PRS+CNCM I-5388 Δgad mutant+free glutamate” group (black rhombus, solid line)). *P<0.05; **P<0.01; ****P<0.0001 vs. basal values for animals treated with vehicle. ⁺P<0.05; ⁺⁺P<0.01 vs. values for stressed animals treated with vehicle.

### DETAILED DESCRIPTION

### Example 1: Physiological Characterization of Bacterial Suspensions of Interest

### 1. Materials and Methods

### 1.1 Bacterial Strains, Medium and Culture Conditions:

The data set is composed of four strains belonging to the Lactococcus lactis subsp. lactis. The two strains, NCDO2118 and NCDO2727 were isolated from vegetables while CNCM I-5388 and CNCM I-5386 have a dairy origin (raw milk and whey respectively).

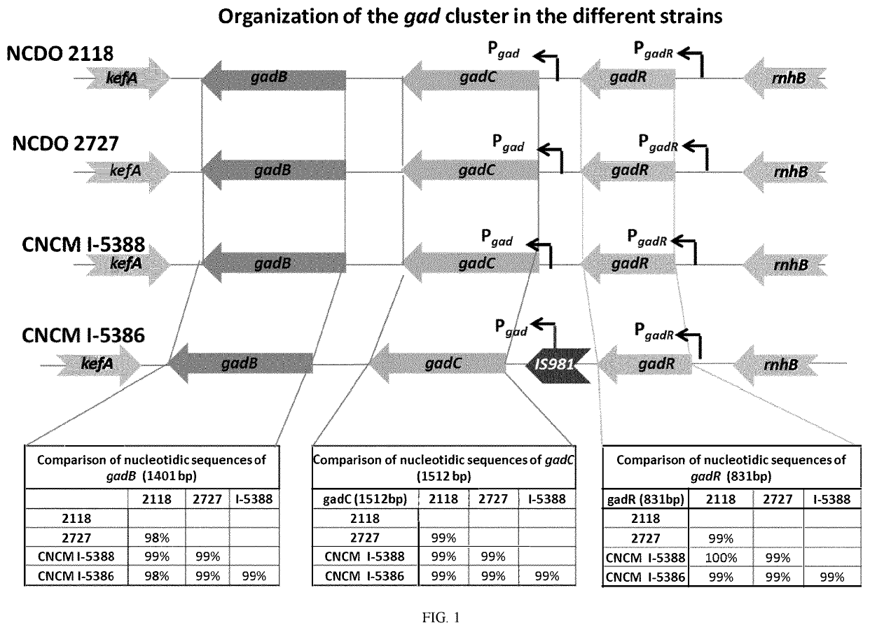

In these strains, the gad cluster involved in GABA biosynthesis is located on the chromosome between kefA and rnhB genes (FIG. 1). The gad cluster is composed of gadR, gadC and gadB encoding respectively the operon regulator, glutamate/GABA antiporter and glutamate decarboxylase. The same cluster organization is retained in these strains except for CNCM I-5386. Indeed, a copy of the insertion sequence IS981 has been found in the promoter of the gadCB operon. This insertion sequence contains outward-35 promoter component and its insertion at the correct distance from the native—10 box can modify the strength of the promoter and its regulation. The nucleotide sequences of gadR, gadC and gadB genes are highly similar in the 4 strains with more than 98% identity (FIG. 1).

Bacterial cultures were performed in duplicate in 2-L Biostat B-plus bioreactor (Sartorius, Melsungen, Germany) in medium M17 (Table 1) supplemented with 55 mM (8 g/L) free glutamate, 29 mM (5 g/L) arginine, 250 mM (45 g/L) glucose and 300 mM NaCl. Cultures were incubated at 30° C.

TABLE 1
Composition of medium M17.

| Ingredients | g/L |
|---|---|
| Tryptone | 2.5 |
| Peptic digest of animal tissue | 2.5 |
| Papaic digest of soybean meal | 5 |
| Meat extract | 5 |
| Yeast extract | 2.5 |
| Ascorbic acid | 0.5 |
| Magnesium sulphate | 0.5 |
| Sodium glycerophosphate | 19 |

Fermentations were carried out under oxygen-limiting conditions, with air in the gaseous phase but without air bubbling. pH was maintained at 6.6 by KOH addition for 8 h, then pH was modified and regulated at 4.6.

Culture was inoculated with cells from pre-cultures grown in Erlenmeyer flask on similar medium, harvested during the exponential phase and concentrated in order to obtain an initial optical density (OD) at 580 nm of 0.25 in the fermenter.

Bacterial growth was estimated by spectrophotometric measurements at 580 nm (Libra S11, Biochom, 1 Unity of absorbance is equivalent to 0.3 g/L).

Samples were collected every 30 min for HPLC measurement of GABA concentration in the growth medium.

Cellular samples for oral administration were prepared as follows. Cells were harvested before the pH modification (i.e. at 7 h for NCDO2118 and NCDO2727).

The culture volume required for approximately 3×10¹¹ cells was centrifuged at 5000×g and 4° C. for 10 min to pellet the bacterial cells.

Cells were washed twice with 0.9% NaCl and suspended in 0.9% NaCl containing 15% glycerol to a final concentration of 10×10⁹ cfu/mL and stored at −20° C. until use.

### 1.2 GABA Extraction and Quantification:

GABA concentration in culture supernatant or reaction mixture of enzyme assay was measured by HPLC (Agilent Technologies 1200 Series, Waldbronn, Germany).

Prior to HPLC, proteins were precipitated by adding four volumes of methanol to one volume of sample. The mixture was centrifuged and the supernatant kept for HPLC analysis. Amino acids were automatically derived with OrthoPhtalic Aldehyde (OPA) and 9-fluorenylmethyl-chloroformate (FMOC-C1). The derivatives were separated on Hypersil AA-ODS column (Agilent Technologies) at 40° C. by a linear gradient of acetate buffer (pH 7.2) with triethylamine (0.018%), tetrahydrofuran (0.3%) and acetonitrile. A diode array detector, at 338 nm for OPA derivatives and 262 nm for FMOC derivatives, was used.

### 1.3 Glutamate Decarboxylase (GAD) Activity:

50 mg of bacterial cells were harvested at 7 h of culture. Cells were washed twice with 0.2% KCl and suspended in sodium acetate buffer (100 mM, pH 4.6) containing 4.5 mM MgCl₂, 22% glycerol and 1.5 mM DTT. Cells were disrupted by sonication (four cycles of 30 s and 60 s spaced out, 6.5 m/s) and kept on ice during the treatment. Cell debris were removed by centrifugation for 15 min at 10,000 g and 4° C.

The supernatant was used for enzyme assays, and the protein concentration of the extract was determined by the Bradford method with bovine serum albumin as the standard.

Enzyme assay was realized with 0.5 mL of substrate solution, consisting of 20 mM sodium glutamate, 2 mM pyridoxal phosphate (PLP) incubated at 30° C. then mixed with 0.5 mL supernatant. Every 30 min until 4 h, 100 μL were sampled and inactivated by boiling for 5 min to stop the decarboxylation reaction. Reaction mixtures were subsequently analyzed for the presence of GABA using HPLC.

The GAD activity was measured at 7 h and neutral pH condition.

One unit of enzyme activity was defined here as the amount of enzyme which converted 1 μmole of glutamate per min and per mg of protein.

### 2. Results

### 2.1 In Vitro GABA Production:

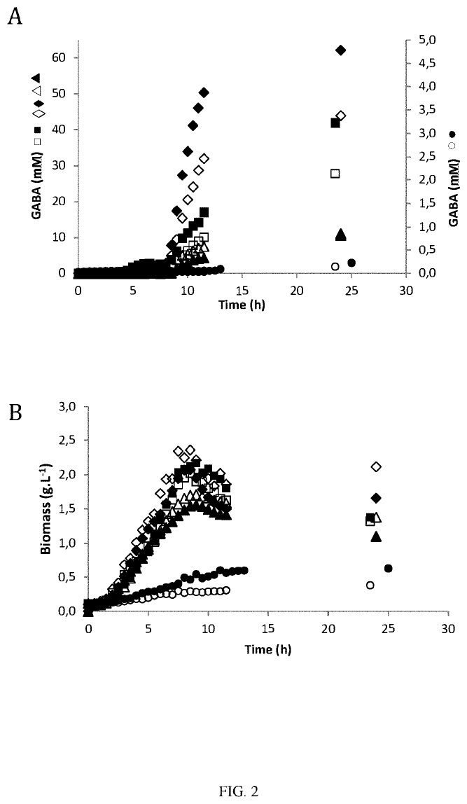

The biomass and GABA concentrations were measured all along the culture for NCDO2118, CNCM I-5388, CNCM I-5386 and NCDO2727 strains (FIG. 2).

In the first 8 hours, the bacterium NCDO2118 as well as CNCM I-5388, CNCM I-5386 grew fast (growth rate around 0.7-0.8 h⁻¹) at pH 6.6 compared to NCDO2727. GABA was accumulated at low levels for all strains. Then at 8 hours, the pH was lowered and controlled to 4.6, growth stopped thereafter and biomass decreased. This loss in cell viability was confirmed by plating.

Despite the growth arrest, the production of GABA by NCDO2118 strain or CNCM I-5388 and CNCM I-5386 continued with increased GABA production rate (Table 2). The maximal GABA concentration reached 60 mM at 24 hours for CNCM I-5388. Similar results were obtained with a second culture in bioreactor (independent duplicate). GABA production of 40-60 mM corresponds to the complete bioconversion of initial concentration of free glutamate into the bioreactor.

Despite the strong similarity of the gad operon sequence and organization in all the four strains, GABA production pattern of the NCDO2727 strain characterized under similar culture conditions was very different with a very weak amount of GABA produced since GABA concentration is always below 0.2 mM (FIG. 2A) and GABA production rate is low (Table 2).

TABLE 2
Specific rates of GABA production calculated between 7 and 11.5 hours (mmole/g cell
dry weight/h) during growth of L. lactis NCDO2118, CNCM I-5388, CNCM I-5386 and
NCDO2727 in bioreactor (in duplicates).

| | NCDO2118 | NCDO2727 | CNCM I-5388 | CNCM I-5386 |
|---|---|---|---|---|
| μmax (h⁻¹) | 0.71 ± 0.05 | 0.21 ± 0.04 | 0.81 ± 0.08 | 0.71 ± 0.05 |
| νₘₑₐₙ GABA | 2.86 ± 0.39 | 0.02 ± 0.04 | 6.70 ± 2.67 | 1.09 ± 0.27 |
| (mmole/g/h) | | | | |

### 2.2 In Vitro GAD Activity:

High GAD activity was obtained in the NCDO2118 strain consistently with its high GABA production ability, while in the NCDO2727 strain, the amount was extremely low (Table 3). Intermediary level of GAD activity was obtained for CNCM I-5386 strain while a very high activity was measured for the CNCM I-5388 strain. One can notice however that, under neutral pH condition at 7 hours, the GAD activity in the NCDO2118 strain, CNCM I-5388 or CNCM I-5386 was high despite the low GABA production accumulated in the bioreactor, suggesting that the enzymatic equipment for GABA production was already expressed in cells but not yet active.

TABLE 3
Specific GAD activity (μmole/min · mg of protein) of the L. lactis strains after 7 hours
of growth in M17 supplemented with free glutamate (8 g/L), arginine (5 g/L), and NaCl (300
mM) (n = 3 for each of the two cultures replicates in bioreactor).

| | NCDO2118 | NCDO2727 | CNCM I-5388 | CNCM I-5386 |
|---|---|---|---|---|
| GAD 7 hours | 42.7 ± 5.9 | 0.6 ± 0.4 | 826.2 ± 93.0 | 20.7 ± 4.3 |

### Example 2: Effect of L. lactis Strains of Interest in a Stress Model in Rat (Model IBS)

### 1. Materials and Methods

### 1.1 Animals and Surgical Procedure:

Adult female Wistar rats (200-225 g) were purchased from Janvier Labs (Le Genest St Isle, France) and individually housed in polypropylene cages under standard conditions (temperature 22±2° C. and a 12-h light/dark cycle). Animals were allowed free access to water and food (standard pellets 2016, Envigo RMS SARL, Gannat, France). All experiments were approved by the Local Animal Care and Use Committee, in compliance with European directive 2010/63/UE.

Under general anesthesia by intraperitoneal administration of 0.6 mg/kg acepromazine (calmivet, Vetoquinol, Lure, France) and 120 mg/kg ketamine (Imalgene 1000, Merial, Lyon, France), animals were prepared for abdominal striated muscle electromyography (EMG) according to a previously described technique (Morteau et al., 1994).

Three pairs of NiCr wire electrodes (60 cm length and 80 μm diameter) were implanted bilaterally in the striated muscles at 2 cm laterally from the midline. The free ends of electrodes were exteriorized on the back of the neck and protected by a glass tube attached to the skin.

### 1.2 Colorectal Distension Procedure and EMG Recordings:

Female rats were accustomed to being in polypropylene tunnels (20 cm length and 7 cm diameter), where they could not move, escape or turn around, for several days before colorectal distension (CRD) in order to achieve familiarization with that environment.

A 4-cm long latex balloon, fixed on rigid catheter taken from an embolectomy probe (Fogarty), was used.

CRD which is a mechanical stimulus was performed after insertion of the balloon in the rectum at 1 cm from the anus. The tube was fixed at the basis of the tail. Isobaric distensions of the colon were performed from 0 to 60 mmHg by connecting the balloon to Distender Series IIR Barostat (G & J Electronics Inc, Toronto, Canada).

The first distension was performed at a pressure of 15 mmHg and an increment of 15 mmHg was added at each following step, until a maximal pressure of 60 mmHg was reached, each distension step lasting 5 min.

The striated muscle spike bursts, related to abdominal cramps, were recorded on an electroencephalograph machine (Mini VIII, Alvar, Paris, France) from implanted electrodes, using a short time constant (0.03 s) to remove low-frequency signals (<3 Hz). EMG recordings started 7 days after surgery.

The number of contractions for a period of 5 minutes represents the intensity of visceral pain.

### 1.3. Stress Procedure:

All stress sessions were performed at the same time of day (between 10 am and 12 noon) to minimise any influence of circadian rhythm.

Partial restraint stress (PRS), a relatively mild non-ulcerogenic model of acute stress, was performed as previously described (Williams et al., 1988).

Female rats were sedated with diethyl-ether and their fore shoulders, upper forelimbs and thoracic trunk were wrapped in a confining harness of paper tape to restrict, but not prevent, body movements.

Animals were then placed in their home cage for 2 h.

### 1.4. Experimental Protocol:

Four series of experiments, based on a 10-day treatment by oral gavage, were conducted using, for each series, three groups of 7 to 12 female rats equipped for EMG.

Rats were given L. lactis NCDO2118 or NCDO2727 by gavage once daily for 10 days. Washed bacterial cells (10⁹ cfu per day) were used in order to minimize the amount of GABA initially administered to rats. Unless otherwise stated, free glutamate (0.2% (w/v)) was added to the gavage mixture to favor in vivo GABA production.

For all conditions, basal abdominal response to CRD was recorded on Day 9 of the treatment and PRS-induced visceral hypersensitivity to CRD recorded on Day 10 of the treatment.

CRD measurements were performed 20 minutes after the 2 h PRS session.

On vehicle-treated animals, free glutamate (0.2% (m/v)) is verified as having no effect per se on basal and stress-induced colorectal sensitivity.

### 1.5. Statistical Methods:

For animal experiments, data are reported as the means±standard errors of the means (SEM). The software GraphPad Prism 6.0 (GraphPad, San Diego, CA) was used for statistical analysis. One-way ANOVA, followed by Tukey's Multiple Comparison test, was performed to compare data between the different groups of animals. Statistical significance was accepted at P<0.05.

### 2. Results

### 2.1. Effect of Free Glutamate on Basal and Stress-Induced Colorectal Sensitivity

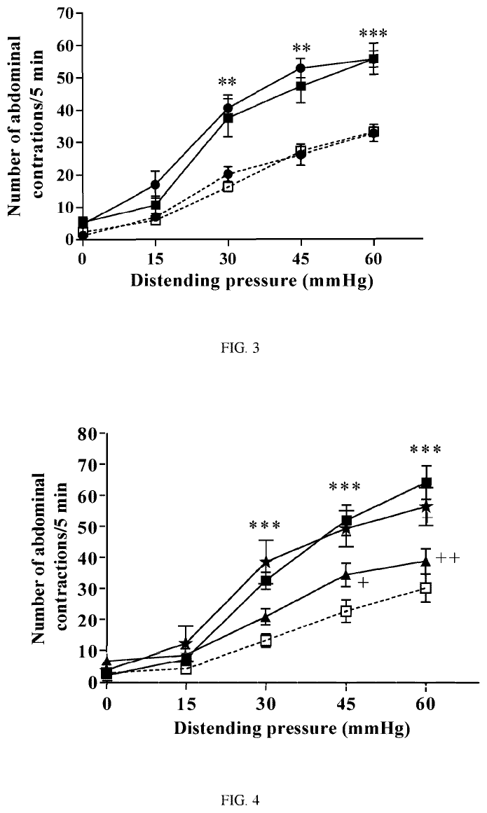

In rats treated with the vehicle under basal conditions (i.e. before PRS), gradual colorectal distension (CRD) increased the frequency of abdominal contractions in a pressure-dependent manner (FIG. 3).

Compared with vehicle, administration of free glutamate did not affect the abdominal response to CRD (FIG. 3).

In vehicle-treated rats, a 2-h PRS significantly increased the number of abdominal contractions in comparison with basal conditions; administration of free glutamate did not modify the visceral hypersensitivity response to CRD measured after the PRS session (FIG. 3).

Thus, free glutamate had no effect per se on basal and stress-induced colorectal sensitivity.

2.2. Effect of the L. lactis NCDO2118 on Visceral Hypersensitivity

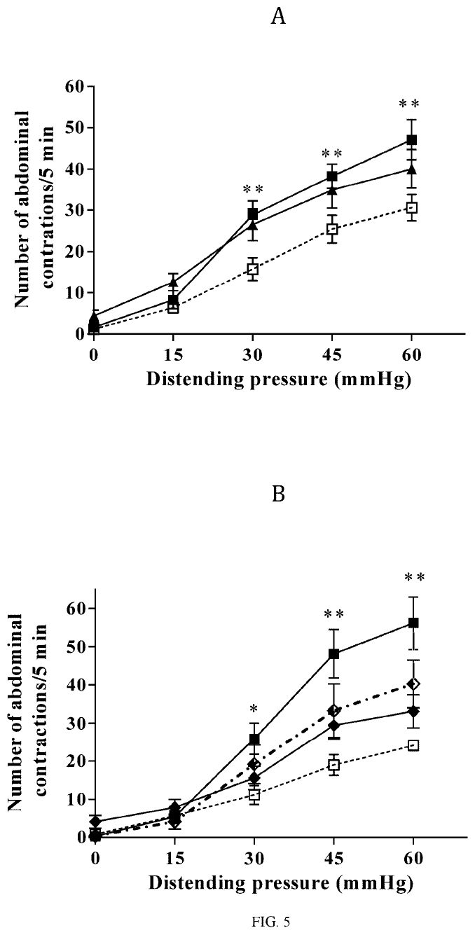

The influence of L. lactis NCDO2118 and NCDO2727 on stress-induced visceral hypersensitivity to CRD is shown in FIG. 4.

In vehicle-treated rats, a 2-h PRS significantly increased the number of abdominal contractions compared to basal conditions for all the pressures of distention applied from 30 mmHg (P<0.001, FIG. 4).

Importantly, a 10-day oral administration of the L. lactis NCDO2118 suppressed the PRS-induced enhancement of abdominal contractions (P<0.01 for a distension pressure of 60 mmHg, FIG. 4), restoring a quasi-basal sensitivity to CRD.

In contrast, oral administration of the L. lactis NCDO2727 had no effect on stress-induced visceral hypersensitivity (FIG. 4).

In conclusion, acute restraint stress induced colonic hypersensitivity to distension in rats and this hypersensitivity phenotype was reversed by L. lactis NCDO2118 but not by L. lactis NCDO2727 after a 10-day daily oral administration (10⁹ cfu per day).

### Example 3: The Prevention of the Stress-Induced Visceral Hypersensitivity by L. lactis NCDO2118 is Due to its Ability to Deliver GABA In Vivo

### 1. Materials and Methods

### 1.1. Bacterial Strains

The strains NCDO2118 and NCDO2727 were used.

L. lactis NCDO2118 AgadB was constructed by double crossing over in the chromosome.

The GAD encoding gene gadB was deleted in order to interrupt the GABA pathway.

Two fragments of 829 and 981 bp were PCR amplified just upstream and downstream of the gadB coding sequence, respectively, with NCDO2118 chromosome as a matrix. Primers are listed in Table 4. The two fragments were fused by overlapping PCR. The pGhost9 vector was PCR amplified and linked to the fused fragments using Gibson assembly method (New Englang Biolabs). Resulting plasmid was verified by sequencing and introduced in L. lactis NCDO2118. Chromosomal deletion of gadB was then obtained by double crossing over as previously described (Maguin et al., 1996). Deletion of gadB sequence into the chromosome was verified by PCR.

The culture conditions of the strains are the same as the one described in Example 1.

TABLE 4
Primers used for the inactivation of gadB in L. lactis NCDO2118.

| | | SEQ ID | |
|---|---|---|---|
| Primer name | 5′-3′ sequence | NO: | Use for: |
| 821-GBgadCR | GGAATTCGATTTAGATGC | 1 | Amplification of |
| | CATAGGAGGATTTTC | | gabB upstream |
| 894-GBgadC2F | GATGAATATCGTACATCC | 2 | sequence |
| | TCCAATTTTTTAATG | | |
| 892-GBkefA2F | AAGCTTGATAAAACAAGA | 3 | Amplification of |
| | AAATATTCATGAAATTCA | | gabB downstream |
| | G | | sequence |
| 893-GBkefA2R | GGAGGATGTACGATATTC | 4 | |
| | ATCTTAAGAAAAATCAAA | | |
| | AGC | | |
| 822-GBpGhost9EVF | CATCTAAATCGAATTCCT | 5 | Amplification of |
| | GCAGCCCG | | pGhost9 backbone |
| 891-GBpGh9EV2R | TCTTGTTTTATCAAGCTTA | 6 | |
| | TCGATACCGTC | | |
| 898-amt | TTGGATTAGCTGCGGCAT | 7 | Verification of |
| GadB(DCO) | ATTTTATCG | | gabB deletion onto |
| 899-avl GadB(DCO) | CCTTGTTGACCATAATGC | 8 | the chromosome |
| | AAAGCAGGT | | |

### 1.2. Sequence Analysis of Gad Operon:

In the NCDO2118 strain, the gad operon sequence was extracted from the chromosome sequence deposited in NCBI-GenBank database under the accession number CP009054.

To amplify the gad operon in NCDO2727 strain, two primers were used, GadSeq_F (5′-TCCAGAAATAACAGCTACATTGACATAATG-3′) and GadSeq_R (5′-TAACAGCCCCA-TTATCTAAGATTACTCC-3′). The amplification was carried out with 20 ng of genomic DNA in 25-μL reaction mixture using the Q5 High-Fidelity DNA polymerase (NEB) and according to the manufacturer's recommendation. PCR conditions consisted of an initial denaturation step of 3 min at 98° C., followed by 30 cycles of 98° C. for 10 sec, 64° C. for 30 sec, and 72° C. for 3 min and a final elongation step of 2 min at 72° C.

Then, the amplicon was purified using the QIAquick gel extraction kit (QIAgen).

The sequence was performed by Eurofins Genomics with a set of primers listed in Table 5. The sequence of the operon has been deposited in NCBI-GenBank database under the accession number MK225577.

Sequences of gad operon were compared by Blast alignment algorithm.

TABLE 5
Primers used for the sequencing of gad operon in L.
lactis NCDO2727.

| Primer name | 5′-3′ sequence | SEQ ID NO: |
|---|---|---|
| GabSeq_2_F | AAAATATAGAAGGAGACTATTGCAAATA | 9 |
| | GC | |
| GabSeq_2_R | AAAAATTAATGGCCATCGTTGGTAGTTCT | 10 |
| | C | |
| GabSeq_3 F | TCTGTGCAGCAGAAATGGCGACGGTTGA | 11 |
| | A | |
| GabSeq_3_R | TCCCCATAAATTTTTCTTTTTCACTCGCAT | 12 |
| GabSeq_4_F | CGGTTATTCCTCAAAAAGACTTATCATTA | 13 |
| | A | |

### 2. Results

With this deletion, and under the same culture conditions, the mutant could grow similarly to the wild type (growth rate of 0.65 h⁻¹). Under these conditions however, no GABA was produced (<0.29 mM at 24 h) and no GAD activity detected at 7 h.

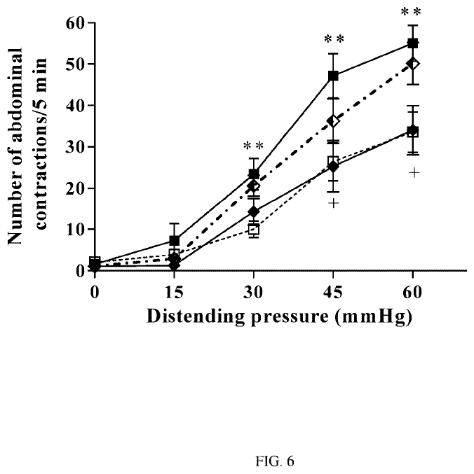

The beneficial effect of L. lactis NCDO2118 treatment on stress-induced visceral hypersensitivity (P<0.001) was reversed when animals received the NCDO2118 ΔgadB isogenic strain (FIG. 6, P<0.05 NCDO2118 ΔgadB-vs. NCDO2118-treated animals).

### Example 4: Comparison of the Effects Obtained on Visceral Hypersensitivity with L. lactis Strains of Interest in a Stress Model in Rat (Model IBS)

### 1. Materials and Methods

The preparation of the animals is the same as the one described in Example 2.

Rats were given L. lactis NCDO2118 or CNCM I-5388 by gavage once daily for 4 or 9 days. Washed bacterial cells (10⁹ cfu per day) were used in order to minimize the amount of GABA initially administered to rats. Unless otherwise stated, free glutamate (0.2% (w/v)) was added to the gavage mixture to favor in vivo GABA production.

### 2. Results

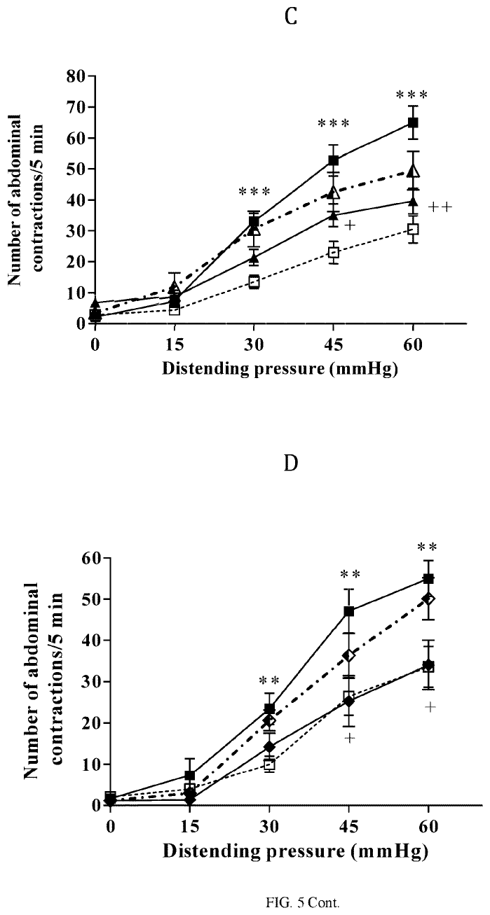

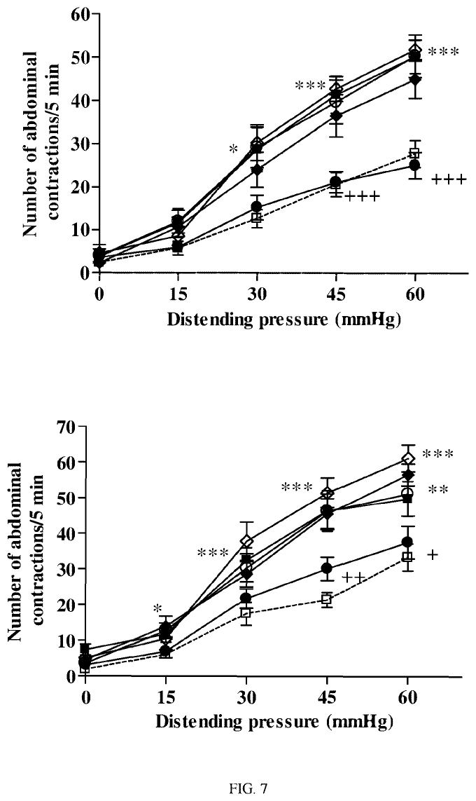

The influence over time of L. lactis NCDO2118, CNCM I-5388 and CNCM I-5388 Δgad on stress-induced visceral hypersensitivity to CRD is shown in FIGS. 5 and 7.

CNCM I-5388 and NCDO2118 in the presence of glutamate reduce the visceral hypersensitivity to the stress.

### REFERENCES

Ait-Belgnaoui A. et al., Pain. 2005; 113:141-7.
Bouin M. et al., Dig. Dis. Sci. 2001; 46:2542-2548.
Camilleri M. et al., Clin. Gastroenterol. Hepatol. 2008; 6:772-781.
Camilleri M et al., Am J Physiol Gastrointest Liver Physiol. 2012; 303: G775-85.
Chang L. et al., Gastroenterology. 2011; 140:761-765.
Coculescu E. et al., J. Med. Life. 2014; 7:512-515.
Craig O. et al., Curr Opin Gastroenterol. 2018; 34:50-56.
Dean B. B. et al., Am J Manag Care. 2005; 11: S17-26.
de Castro L A. et al., Pain Med. 2014; 15:2164-5.
Didari T. et al., World. J. Gastroenterol. 2015; 21:3072-3084.
Ford A. C. et al., Am. J. Gastroenterol. 2014; 109:1547-1561.
Gue M. et al., Neurogastroenterol Motil. 1997; 9(4): 271-9.
Hungin A. P. et al., Aliment. Pharmacol. Ther. 2013; 38:864-886.
Keszthelyi D. et al., EJP. 2012; 16:1444-1454.
Kuiken S. D. et al., Aliment. Pharmacol. Ther. 2005; 22:157-164.
Laroute V. et al., Microorganisms. 2017; 5.
Maguin E. et al., J. Bacteriol. 1996; 178:931-935.
Mayer E. A. et al., Gastroenterology. 1994; 107:271-293.
Mayer E. A. et al., Gut. 2008; 57:384-404.
Mazurak N. et al., J. Neurogastroenterol. Motil. 2015; 21:471-485.
Mills S. et al., Int. J. Dairy Technol. 2010; 63:149-170.
Miziara I D. et al., J. Psychosom. Res., 2009; 62:443-448.
Moayyedi P. et al., Gut. 2010; 59:325-332.
Morteau O. et al., Dig. Dis. Sci. 1994; 39:1239-1248.
Nomura M. et al., Int. J. Syst. Bacteriol. 1999; 49:163-166.
Pérez-Berezo T. et al., Nat. Commun. 2017; 8, 1314.
Pokusaeva K. et al., Neurogastroenterol. Motil. 2017; 29, e12904.
Posserud I. et al., Gastroenterology. 2007; 133:1113-1123.
Radziwill-Bienkowska et al., Appl. Microbiol. Biotechnol. 2016; 100:9605-9617.
Sun Y. et al., Chin J Dig Dis. 2006; 7:149-55.
Sun A. et al., J. Oral Pathol. Med., 2013; 42:649-655.
Wang Y. et al., J. Microbiol. Methods. 2011; 86:390-392.
Williams C. L. et al., Am J Physiol. 1987; 253: G582-6.
Williams C. L. et al., Gastroenterology. 1988; 94:611-621.
Zhang C. et al., ISME J. 2016; 10:2235-2245.
Zhou Q. et al., Pain. 2010; 148:454-461.

1. A method of treating stress or anxiety in a subject comprising administering to the subject a composition comprising a Lactococcus lactis (L. lactis) strain, or a cell fraction thereof, wherein the L. lactis strain is CNCM I-5388, CNCM I-5386, or a combination thereof.

2. The method of claim 1, wherein the L. lactis strain comprises:
(a) living cells;
(b) dead cells;
(c) a lysate; or
(d) a cell fraction wherein a membrane fraction has been separated from the total lysate.

3. The method of claim 1, wherein the composition comprises:
(a) at least 10⁶ colony forming units (cfu) of said L. lactis strain;
(b) at least 10⁷ cfu of said L. lactis strain;
(c) at least 10⁸ cfu of said L. lactis strain; or
(d) at least 10⁹ cfu of said L. lactis strain.

4. The method of claim 1, wherein the L. lactis strain is CNCM I-5388.

5. The method of claim 1, wherein the composition further comprises free glutamate.

6. The method of claim 5, wherein the composition comprises about 4 mg free glutamate per 10⁹ cfu of L. lactis.

7. The method of claim 5, wherein the free glutamate is administered in an amount corresponding to about 10 to about 20 mg/kg body weight.

8. The method of claim 1, wherein the composition comprises a pharmaceutical composition, a nutritional composition, or a food product.

9. The method of claim 1, wherein the composition comprises a liquid, a solid, a dry powder, a tablet, a capsule, a syrup, an effervescent composition, or a spray.

10. The method of claim 1, wherein the subject has burning mouth syndrome or suffers from visceral pain.

11. The method of claim 1, wherein the stress or anxiety comprises psychological stress or anxiety.

12. A method of preventing or treating abdominal or visceral pain in a subject comprising administering to the subject a L. lactis strain, or a cell fraction thereof, wherein the L. lactis strain is CNCM I-5388, CNCM I-5386, or a combination thereof.

13. The method of claim 12, wherein the subject has burning mouth syndrome, an injury, an infection, an inflammation, a disease, a chronic abdominal and pelvic pain disorder, or irritable bowel syndrome.

14. The method of claim 12, wherein the L. lactis strain comprises:
(a) living cells;
(b) dead cells;
(c) a lysate; or
(d) a cell fraction wherein a membrane fraction has been separated from the total lysate.

15. The method of claim 12, wherein the composition comprises:
(a) at least 10⁶ colony forming units (cfu) of said L. lactis strain;
(b) at least 10⁷ cfu of said L. lactis strain;
(c) at least 10⁸ cfu of said L. lactis strain; or
(d) at least 10⁹ cfu of said L. lactis strain.

16. The method of claim 12, wherein the L. lactis strain is CNCM I-5388.

17. The method of claim 12, wherein the composition further comprises free glutamate.

18. The method of claim 17, wherein:
(a) the composition comprises about 4 mg free glutamate per 10⁹ cfu of L. lactis; and/or
(b) the free glutamate is administered in an amount corresponding to about 10 to about 20 mg/kg body weight.

19. The method of claim 12, wherein the composition comprises a pharmaceutical composition, a nutritional composition, or a food product.

20. The method of claim 12, wherein the composition comprises a liquid, a solid, a dry powder, a tablet, a capsule, a syrup, an effervescent composition, or a spray.
